(19) United States
(12) Patent Application Publication
(10) Pub. No.: US 2018/0348198 A1
(43) Pub. Date: Dec. 6, 2018

(54) SYSTEMS AND METHODS FOR VERIFICATION VALUE OF USER ENTERED VALUES RESULTING FROM POINT OF CARE TESTING USING A METER

(71) Applicant: Polymer Technology Systems, Inc., Indianapolis, IN (US)

(72) Inventor: Jonathan A. Broadwell, Beech Grove, IN (US)

(73) Assignee: Polymer Technology Systems, Inc., Indianapolis, IN (US)

(21) Appl. No.: 15/992,569

(22) Filed: May 30, 2018

Related U.S. Application Data
(60) Provisional application No. 62/516,006, filed on Jun. 6, 2017.

(51) Int. Cl. G01N 33/49; G01N 33/487; A61B 5/145

(52) U.S. Cl. CPC G01N 33/492; A61B 5/14532; G01N 33/48792

(57) ABSTRACT

A method of verifying test data generated by a meter includes testing the sample with the meter. The meter further generates and displays a first analyte level, and calculates and displays a First Verification Value. The method further includes displaying a user interface using a computing system for receiving a user input and receiving the user input including the first analyte level and the First Verification Value. The receiving computing system calculates a Second Verification Value from the entered Result Data. The method further includes verifying with the computing system that the user input was correctly entered by comparing the entered First Verification Value with the calculated Second Verification Value and provides an indication to the user as to whether the user input was correctly entered. The meter may additionally generate additional analyte levels and the Verification Values include the additional analyte levels times corresponding, predetermined multipliers.

### CROSS-REFERENCE TO RELATED APPLICATION

This application claims the benefit of U.S. Provisional Application No. 62/516,006 filed Jun. 6, 2017, which is hereby incorporated by reference.

### BACKGROUND

Point of Care (“POC”) and home testing for various blood analytes and other detectable metrics in bodily fluids is desirable for patient and doctor. In many scenarios, patients and doctors can determine critical features related to the immediate and long term health of patients with simple test strips used with meters or other point of care analysis devices. These devices are usually simple to use and provide results within minutes. As part of the generation of POC results, many times users enter data output by a meter into another electronic device, typically a computer. During the entry, there is the possibility for user error which will result in the entered data being essentially unusable. After entry, typically the users forget or discard the results appearing on the meter, so the data will be unrecoverable. Additionally, at health fairs or similar events, administrators may enter numerous sets of data and may not realize that a transcription error has occurred.

### BRIEF SUMMARY

In one embodiment, a system for verifying testing data generated by a meter includes a meter, the meter executing code to analyze a bodily fluid and generate a first analyte level in the bodily fluid, the meter having a display displaying the first analyte level and a First Verification Value. The system further includes a computing system executing code for displaying an interface for receiving a user input including an entered first analyte level and an entered First Verification Value.

The computing system further executes code for analyzing the entered first analyte level and the entered First Verification Value and for determining whether the user input has been correctly entered into the computing system by comparing the entered First Verification Value to a calculated Second Verification Value. The computing system further provides an indication concerning correct entry of the user input. Optionally, the Verification Values are set to the first analyte level times a predetermined multiplier. Alternatively, the meter additionally generates a second analyte level and the Verification Values are set to the first predetermined analyte level times a first multiplier plus the second analyte level times a second predetermined multiplier.

In one alternative, the first multiplier and the second multiplier are different. In another alternative, the first multiplier and the second multiplier are prime integers. Optionally, the first analyte level and the second analyte level are selected from the group consisting of an HDL level, an LDL level, a total cholesterol level, a glucose level, and a triglycerides level. Alternatively, the meter additionally generates a third analyte level and the third analyte level times a third predetermined multiplier is incorporated in the Verification Values.

In one embodiment, a method of verifying testing data generated by a meter includes providing a sample to be tested by a meter. The method further includes testing the sample with the meter. The method further includes generating a first analyte level and a First Verification Value with the meter. The method further includes displaying the first analyte level and the First Verification Value with the meter. The method further includes displaying a user interface using a computing system for receiving a user input including an entered first analyte level and an entered First Verification Value.

The method further includes receiving the user input including the entered first analyte level and the entered First Verification Value at the computing system. The method further includes verifying, such as with the computing system, that the user input has been correctly entered by comparing the entered First Verification Value to the calculated Second Verification Value and providing an indication to the user with the computing system as to whether the user input has been correctly entered. Optionally, the Verification Values are set to the first analyte level times a multiplier.

Alternatively, the meter and the computing system separately calculate the Verification Values. In one alternative, the meter additionally generates a second analyte level and the Verification Values are set to the first analyte level times a first predetermined multiplier plus the second analyte level times a second predetermined multiplier. Optionally, the first multiplier and the second multiplier are different. Alternatively, the first multiplier and the second multiplier are prime integers. Optionally, if the indication is that the user input has been entered correctly, then the method further includes storing the user input and/or transmitting the user input to a remote database via the computing system.

In one embodiment, a non-transitory, computer-readable storage device is coupled to one or more processors and has instructions stored thereon which, when executed by the one or more processors, cause the one or more processors to perform operations for verifying testing data generated by a meter. The operations further include displaying a user interface for receiving a user input including an entered first analyte level and an entered First Verification Value. The entered first analyte level and the entered First Verification Value are obtained by a user reading and transcribing data from a meter testing for an analyte and displaying a first analyte level and a First Verification Value to the user.

The instructions further include receiving the user input including the entered first analyte level and the entered First Verification Value at the computing system. The instructions further include verifying, with the computing system, that the user input has been correctly entered by comparing the entered First Verification Value to the calculated Second Verification Value and providing an indication to the user with the computing system as to whether the user input has been correctly entered. Optionally, the Verification Values are set to the first analyte level times a predetermined multiplier. Alternatively, the meter additionally generates a second analyte level and the Verification Values are set to the first analyte level times a first predetermined multiplier plus the second analyte level times a second predetermined multiplier. In one alternative, the meter and the computing system separately calculate the Verification Values. In another alternative, the first multiplier and the second multiplier are different. Optionally, the first multiplier and the second multiplier are prime integers.

### BRIEF DESCRIPTION OF THE DRAWINGS

FIG. 1 shows one embodiment of a system utilizing a checksum verification.

FIG. 2 shows one embodiment of a flow chart for a method for verifying the proper entry of meter result data.

FIG. 3 shows an exemplary interface of a meter generating a Verification Value.

FIG. 4 is a screenshot of a spreadsheet, showing that when the calculated checksum does not match the transcribed checksum then an error result is produced.

### DETAILED DESCRIPTION

Certain terminology is used herein for convenience only and is not to be taken as a limitation on the embodiments of the systems and methods for checksum verification of user entered values resulting from point of care testing using a meter. In the drawings, the same reference numbers are employed for designating the same elements throughout the several figures.

In many embodiments, the system generates a checksum in addition to levels of various analytes. In some embodiments, the checksum is the sum of multipliers times respective one or more analyte levels. In many configurations, the multiplier for each analyte level is different. Other possibilities are available for calculation. However, having a different multipler for each analyte level is advantageous since, even if the correct numbers are entered in the wrong order or for the wrong analyte(s), the error will be detected in most scenarios. Although various scenarios including 3 analytes and other numbers of analytes are discussed herein, any number of analytes may be used, typically those scenarios including 2 or more analytes.

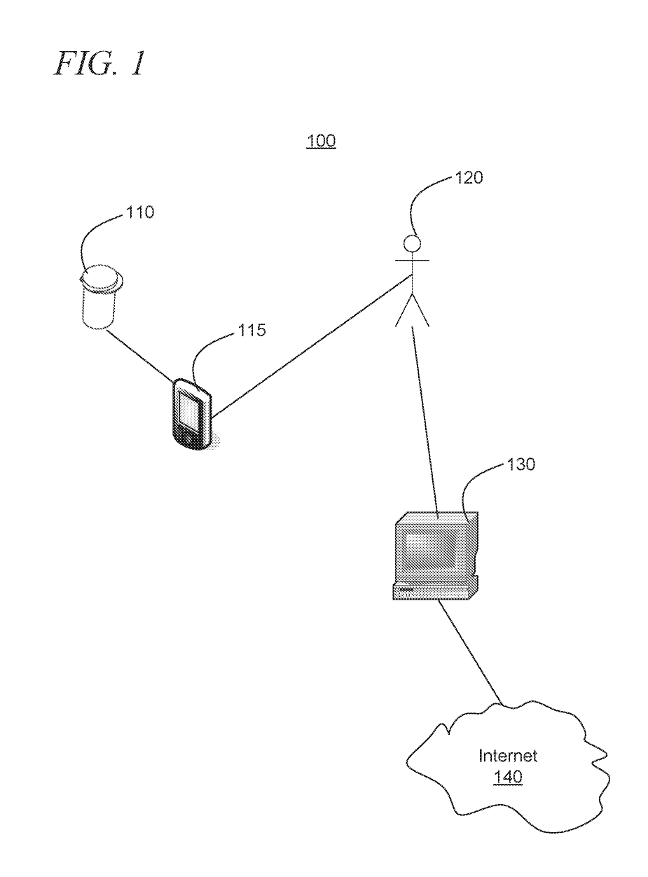

FIG. 1 shows one embodiment of a system utilizing a checksum verification. System 100 includes test strips 110 and a meter 115. A user 120 retrieves one or more test strips 110 and utilizes them in conjunction with meter 115 to test for the level of various analytes, typically in a blood sample or other sample. User 120 may be testing himself or herself or may be testing another individual. Subsequent to testing, meter 115 generates various analyte levels and a calculated verification value or checksum.

User 120 reads the various analyte levels from meter 115 and inputs them into computer 130. Computer 130 is typically running specialized software for recording the analyte levels and potentially other information about the individual being tested. Computer 130 may be a laptop or desktop computer, or may be another computing device such as a tablet, smartphone, etc. Computer 130 may subsequently report results over the Internet 140 or some other computer network.

Computer 130 includes specialized software that presents an interface for the user 120 to enter the results including a verification value (or checksum). Generally, user 120 may imperfectly read or enter the analyte levels observed on meter 115. The computer 130, based on the results and the verification values, determines whether any transcription errors occurred. Further information concerning the generation of the verification values (checksum) is provided below.

Embodiments of the systems and methods described herein can detect errors in manual transcription of data when applied to measured result data. Embodiments can detect any single transcription error and is unlikely to fail to detect multiple transcription errors in the same result data. Embodiments of the systems and methods achieve this result by calculating a verification value or checksum, based on the analyte levels output by the meter. For example, a single blood test might result in measured result data for blood concentration in mg/dL of total cholesterol, HDL cholesterol, LDL cholesterol, Triglycerides, and Glucose.

In order generate a verification value for result data, each type of result data is pre-assigned a fixed multiplier. For example, the following values might be chosen for this example:

TABLE 1
Result Data and Multipliers

| Result Data Type | Multiplier (example) |
|---|---|
| Triglycerides | 5 |
| Glucose | 7 |
| Total Cholesterol | 11 |
| HDL Cholesterol | 13 |
| LDL Cholesterol | 17 |

In order to generate the Verification Value, each type of result data is multiplied by its respective multiplier and added to a sum. For example, a blood test was performed on a patient and the result data was reported as shown in Table 2, and the Verification Value was 4797.

TABLE 2
Verification Value Calculation

| | | | Product | Verification |
|---|---|---|---|---|
| | | | (Result | Value |
| | | | data × | (Sum of |
| Result Data Type | Result Data | Multiplier | Multiplier) | Products) |
| Triglycerides | 58 mg/dL | 5 | 290 | 4797 |
| Glucose | 92 mg/dL | 7 | 644 | |
| Total Cholesterol | 152 mg/dL | 11 | 1672 | |
| HDL Cholesterol | 43 mg/dL | 13 | 559 | |
| LDL Cholesterol | 96 mg/dL | 17 | 1632 | |

In this example, the multiplier values were chosen based on a number of factors. First, a verification value of 4 to 5 digits is desired. This length is short enough to be easily transcribed in a single mental operation. The data types with the largest possible measured values (in this case Triglycerides) are given the smallest multiplier. Multipliers in this example are prime numbers, which reduces the chance that multiple concurrent transcription errors will compensate for one-another and provide a Verification Value which erroneously matches the expected, error-free, Verification Value.

Finally, integers are used. It is not necessary that all numbers involved are integers. However, for non-integer data it is important that rounding rules and tolerances for what defines equality of Verification Values be defined.

In the case of data for blood results, different multiplier values may be assigned if multiple units of measure are provided. For example, multipliers used when blood analyte concentrations are measured in mg/dL would not be ideal for measurements expressed in mmol/L or g/dL.

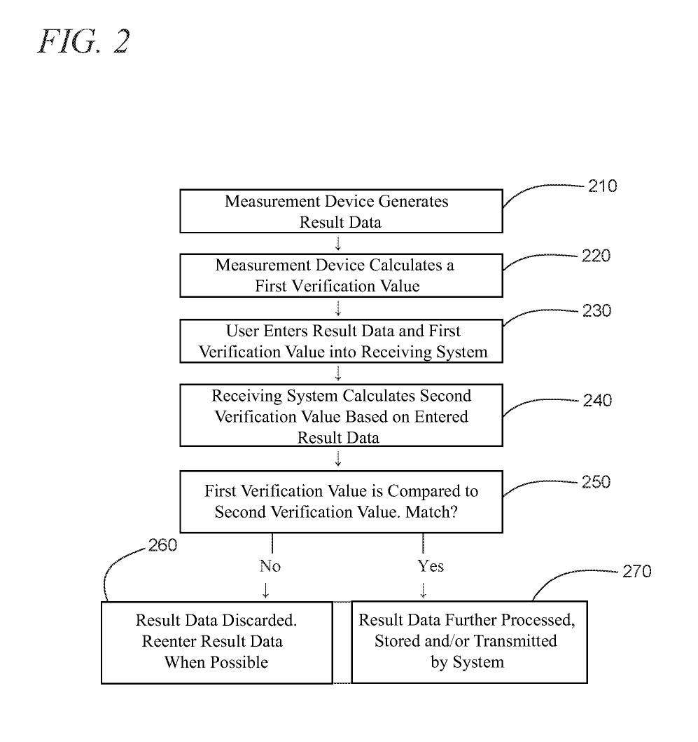

FIG. 2 shows one embodiment of a flow chart for a method for verifying meter data. In step 210, the measurement device generates result data, for example, analyte values. This is typically a meter utilizing a test strip that measures analyte levels in a blood sample. In step 220, the measurement device calculates a First Verification Value. This is performed according to the verification value procedures described above, typically. In step 230, the user reads the analyte values and the First Verification Value from the meter and enters them into the receiving computing system.

In step 240, the computing system calculates a Second Verification Value using the entered Result Data and the predetermined multipliers. The computer system then in step 250 compares the calculated Second Verification Value with the entered First Verification Value. If the Verification Values do not match, then in step 260 there is a transcription error and the user is notified. If the values match, then in step 270 the user is notified and the results may be passed on. Depending on the specifics of the software, interface, or other system, steps 260 and 270 may occur in different ways.

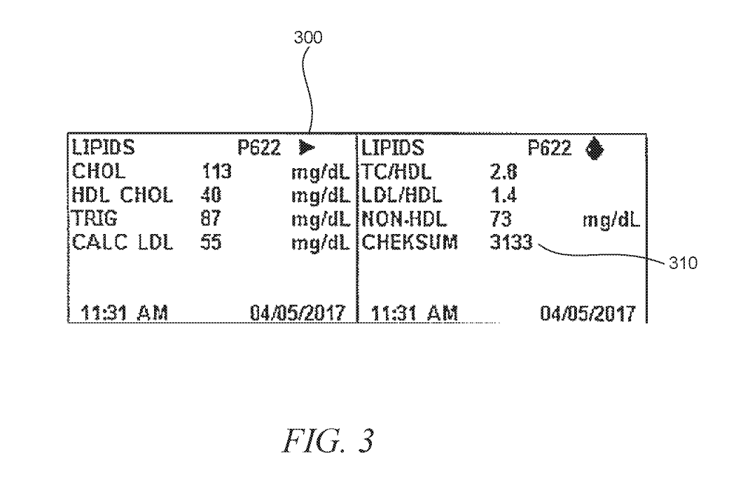

FIG. 3 shows an exemplary interface 300 of a meter generating a checksum (First Verification Value) 310. In the example, a PTS CardioChek Plus meter, available from PTS Diagnostics, was modified to create a First Verification Value based on the analyte levels. The meter output total cholesterol, HDL cholesterol, triglycerides, and LDL cholesterol as well as the First Verification Value. A spreadsheet was created to verify the Verification Value against the Result Data.

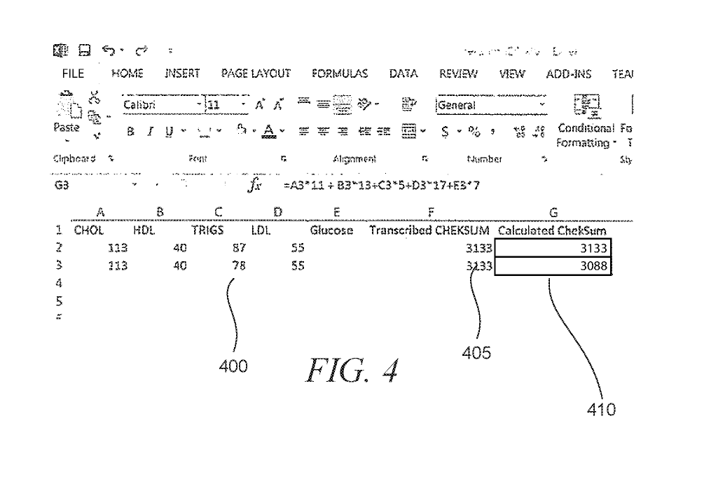

FIG. 4 is a screenshot of a sample spreadsheet. The spreadsheet shows the algorithm for generating the Verification Value from the Result Data. In this example, the formula is A3*11+B3*13+C3*5+D3*17+E3*7. Turning to row 3, the entered numbers were cholesterol—113, HDL—40, triglycerides—78 and LDL—55. Using this entered Result Data, the formula results in a Verification Value of 3,088. However, in comparison it is shown in row 2 that the entered Result Data differed from the actual Result Data in that the incorrect triglycerides value 78 (rather than 87) was entered. This number was transposed by the user when entering the Result Data. Accordingly, the First Verification Value was properly 3133. The transposition of the LDL value caused the Second Verification Value to be calculated as 3088. Consequently, the entered Result Data was determined to be incorrect. When the calculated Second Verification Value 410 (based on the entered Result Data) does not match the First Verification Value 405 (as entered by the User), then an error result is produced. The error is shown as the transposed LDL number 400.

In many embodiments, some measurement equipment provides a high or low limit when indicating that a measurement is out of range. For instance, an analyzer which measures HDL Cholesterol might report “Less than 20 mg/dL” for a patient with particularly low blood concentrations of that substance. In this case, it is important that the Verification Value indicate that an unmeasurably low value has been received. For example, in this case, the numerically reported value accompanying “less than” may have one (1) subtracted from it, and then be rounded down to the next lowest whole integer before being multiplied by the multiplier for that data type. Similarly, values reported along with a “Greater Than” designator may have one (1) added to them, and then be rounded down. It is important to note that this change does not occur to the actual result data, which is still reported as being “Less than” or “Greater Than” a particular value. The change is only to the value which is incorporated into the Verification Value. This allows the Verification Value to differentiate between “<20” and “20”, and assures that the “Less Than” or “Greater Than” marker was appropriately transcribed.

Some measurement equipment may report “Not Available” in place of a result. In this case negative-one (−1) or some other predetermined number is used as the data value, and multiplied by the multiplier for that data type.

An important feature of embodiments of the systems and methods is that transposition of data types (for example, recording LDL Cholesterol values as HDL Cholesterol and vice-versa) is also detected as a transcription error, despite the fact that the result data types do not require recording in a specific order. This is accomplished in embodiments where each analyte has a different multiplier. This differentiates this system from other prior algorithms, such as Cyclic Redundancy Checks, which require data to be in a pre-defined order to detect transposition errors. For instance, as shown in table 2, the multiplier for each analyte is different and therefore, if the right numbers are entered in association with the wrong analytes when transcribing, the Verification Value will be erroneous.

In many embodiments, the simplicity of the systems and methods are also an important feature. The methods may be easily implemented by a technically unsophisticated user through a simple spreadsheet function requiring only addition and multiplication (and in some cases rounding) functions. This distinguishes it from other existing algorithms such as ISBN-10, which require breaking individual numbers into digits before multiplying and calculating a sum.

Comparison of the calculated Second Verification Value with the transcribed First Verification Value may occur, for example, by manual calculation or by calculation in a spreadsheet. That is, the user may calculate the Second Verification Value independently of either the meter or the computing system. However, the calculation and comparison may readily be performed by including the algorithm in custom software in the receiving computing system. In the case where custom software is used, the software may be designed to calculate the Second Verification Value and verify the First Verification Value as data is entered, and prompt the user to re-transcribe if an error is detected, thereby preventing incorrectly transcribed data from being processed.

In many embodiments, parts of the systems and methods are provided in devices including microprocessors. Various embodiments of the systems and methods described herein may be implemented fully or partially in software and/or firmware. This software and/or firmware may take the form of instructions contained in or on a non-transitory computer-readable storage medium. Those instructions then may be read and executed by one or more processors to enable performance of the operations described herein. The instructions may be in any suitable form such as, but not limited to, source code, compiled code, interpreted code, executable code, static code, dynamic code, and the like. Such a computer-readable medium may include any tangible non-transitory medium for storing information in a form readable by one or more computers such as, but not limited to, read only memory (ROM); random access memory (RAM); magnetic disk storage media; optical storage media; a flash memory, etc.

Embodiments of the systems and methods described herein may be implemented in a variety of systems including, but not limited to, smartphones, tablets, laptops, and combinations of computing devices and cloud computing resources. For instance, portions of the operations may occur in one device, and other operations may occur at a remote location, such as a remote server or servers. For instance, the collection of the data may occur at a smartphone, and the data analysis may occur at a server or in a cloud computing resource. Any single computing device or combination of computing devices may execute the methods described.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein, but is intended to cover modifications within the spirit and scope thereof as defined by the appended claims and any and all equivalents thereof. Note that, although particular embodiments are shown, features of each may be interchanged between embodiments.

What is claimed as new and desired to be protected by Letters Patent of the United States is:

1. A system for verifying the accurate entry of test data generated by a meter, the system comprising:
a meter executing code to analyze a bodily fluid and generate a first analyte level in the bodily fluid, the meter generating a first verification value based on the first analyte times a predetermined multiplier, the meter having a display configured to display the first analyte level and the first verification value; and
a computing system executing code for displaying an interface for receiving a user input including an entered, first analyte level and an entered first verification value, the computing system being configured to compute a second, calculated verification value based on the entered analyte level and the predetermined multiplier, the computing system further executing code for comparing the entered first verification value and the calculated second verification value to determine the correctness of the analyte level entered into the computing system, the computing system further providing an indication concerning the correctness of the entered analyte value.

2. The system of claim 1, wherein the meter additionally generates and displays a second analyte level and a second verification value, the first and second verification values further including the second analyte level times a second predetermined multiplier.

3. The system of claim 2, wherein the first multiplier and the second multiplier are different.

4. The system of claim 3, wherein the first multiplier and the second multiplier are prime integers.

5. The system of claim 2, wherein the first analyte level and the second analyte level are selected from the group consisting of an HDL level, an LDL level, a total cholesterol level, a glucose level, and a triglycerides level.

6. The system of claim 2, wherein the meter additionally generates and displays a third analyte level and a third verification value, the verification values further including the third analyte level times a third predetermined multiplier.

7. A method of verifying the entry of test data generated by a meter, the method comprising:
testing a sample with the meter;
generating a first analyte level and a first verification value with the meter;
displaying the first analyte level and the first verification value with the meter;
displaying a user interface using a computing system for receiving a user input including an entered first analyte level and an entered first verification value;
receiving the user input including the entered first analyte level and the entered first verification value at the computing system;
verifying, with the computing system, whether the user input was correctly entered the first analyte value by comparing the calculated second verification value to the entered first verification value; and
providing an indication to the user with the computing system as to whether the first analyte value was correctly entered.

8. The method of claim 7, wherein the first verification value is set to the first analyte level times a predetermined multiplier.

9. The method of claim 8, wherein the meter and the computing system separately calculate the first and second verification values, respectively.

10. The method of claim 8, wherein the meter additionally generates and displays a second analyte level and a second verification value, the verification values including the second analyte level times a second predetermined multiplier.

11. The method of claim 10, wherein the first multiplier and the second multiplier are different.

12. The method of claim 11, wherein the first multiplier and the second multiplier are prime integers.

13. The method of claim 7, wherein if the indication is that the user input has been entered correctly, then the method further comprises:
storing the user input; and
transmitting the user input to a remote database via the computing system.

14. A non-transitory computer-readable storage device coupled to one or more processors and having instructions stored thereon which, when executed by the one or more processors, cause the one or more processors to perform operations for verifying the correctness of entry of test data generated by a meter, the operations comprising:
displaying a user interface for receiving a user input including an entered first analyte level and an entered first verification value, the entered first analyte level and the entered first verification value being received from a user reading and transcribing same from a meter testing for an analyte and displaying the first analyte level and the first verification value to the user;
receiving the user input including the entered first analyte level and the entered first verification value at the computing system;
computing with the computing system a second, calculated verification value based on the entered analyte level and the predetermined multiplier;
verifying, with the computing system, that the user input has been correctly entered by comparing the entered first verification value with the calculated second verification value; and
providing an indication to the user with the computing system as to whether the user input was correctly entered.

15. The non-transitory computer-readable storage device of claim 14, wherein the second verification value comprises the first analyte level times a predetermined multiplier.
